(19) United States
(12) Patent Application Publication
(10) Pub. No.: US 2005/0244977 A1
(43) Pub. Date: Nov. 3, 2005

(54) Adaptive metal films for detection of biomolecules

(76) Inventors: Vladimir P. Drachev, West Lafayette, IN (US); Mark D. Thoreson, West Lafayette, IN (US); Vishal C. Nashine, West Lafayette, IN (US); Meena L. Narsimhan, West Lafayette, IN (US); Eldar N. Khaliullin, West Lafayette, IN (US); Dor Ben-Amotz, West Lafayette, IN (US); Vladimir M. Shalaev, West Lafayette, IN (US); Vincent J. Davisson, West Lafayette, IN (US)

Correspondence Address: INDIANAPOLIS OFFICE 27879;BRINKS HOFER GILSON & LIONE, ONE INDIANA SQUARE, SUITE 1600, INDIANAPOLIS, IN, 46204-2033, US

(21) Appl. No.: 11/087,262

(22) Filed: Mar. 23, 2005

Related U.S. Application Data
(60) Provisional application No. 60/555,944, filed on Mar. 24, 2004.
(60) Provisional application No. 60/569,760, filed on May 10, 2004.
(60) Provisional application No. 60/628,061, filed on Nov. 15, 2004.

(51) Int. Cl. G01N 33/00; G01N 33/543; G01N 33/553

(52) U.S. Cl. 436/86; 436/518

(57) ABSTRACT

An adaptive surface for supporting an analyte to be examined, includes a support layer, a metal island layer, and an adhesive layer by which the metal island layer is attached to the support layer, the adhesive layer and metal island layer being interactive with an analyte containing solution to permit movement of at least some of the islands on the adhesive layer into increasingly close proximity during drying of the analyte solution.

### CROSS-REFERENCE TO RELATED APPLICATIONS

The present application is related to and claims all possible benefits of U.S. provisional application 60/555,944 filed Mar. 24, 2004; 60/569,760 filed May 10, 2004; and 60/628,061 filed Nov. 15, 2004.

### BACKGROUND

1. Technical Field

This invention relates generally to the field of spectroscopy and, more specifically, to surface enhanced Raman spectroscopy (SERS) and fluorescence spectroscopy, and to sample support surfaces for use in such spectroscopy.

2. General Background

Raman scattering is a well known detection method for molecule sensing. Raman spectra can be used to fingerprint molecules of particular interest. Raman spectroscopy can provide important structural information on conformational changes, such as between native and denatured molecules. Raman difference spectroscopy can be used to detect allosteric conformational changes in biomolecules. SERS provides even greater detection sensitivity than conventional Raman spectroscopy. SERS is particularly well suited for the study of biological molecules that have been adsorbed on a metal surface. SERS spectroscopy allows for the detection and analysis of minute quantities of analytes. The large scattering enhancements of SERS permit one to obtain high-quality SERS spectra at sub-monolayer molecular coverage. SERS is sensitive to molecular orientation and to the distance between the molecule of interest and an adjacent metal surface. SERS can be particularly efficient in detecting the conformational state and orientation of molecules since there is generally a preferable orientation of the molecular subunits on a metal surface that can be different for different conformations.

The SERS enhancement mechanism originates in part from the large local electromagnetic fields caused by resonant surface plasmons that can be optically excited at a certain wavelength for metal particles of different shapes or closely spaced groups of particles. For aggregates of interacting particles, which can be structured as fractals, plasmon resonances can be excited in a very broad spectral range. In addition to electromagnetic field enhancement, metal nanostructures and molecules can form charge-transfer complexes that provide further enhancement for SERS. The resulting overall enhancement depends on the particle or aggregate nanostructure morphology. The enhancement can be as high as 10⁵ to 10⁸ for the area-averaged macroscopic signal and as high as 10¹⁰ to 10¹⁵ within the local resonant nanostructures.

A variety of structures have been found to be appropriate for SERS, including roughened metal electrodes, aggregated films, metal particles of different fixed morphology, and semi-continuous films near the percolation threshold. The effects on the metal films due to deposition rate, mass thickness, and thermal annealing have been previously studied. Some work has been done to engineer optimal nanostructures with a controlled particle shape, such as triangles, nanoshells, and even bow-tie like structures. Regular arrangements have been achieved by nanosphere lithography, electron beam lithography and through metal coating of dielectric spheres. Despite the variety of shapes and sizes available, metal nanostructures with a fixed morphology rarely match different analytes or provide the optimal SERS spectra in all cases due in part to the large differences in analyte sizes. Further, many substrates are not biocompatible, yet it is of great interest to study biomolecules in their native forms with functionality preserved. Biomolecular research often involves several washing procedures to remove non-binding or excess agents. This washing can also remove metal nanoparticles that have medium to low adhesion to the substrate. Thus, efforts continue to find even better metal surface shapes, sizes, and other characteristics providing further enhancement of SERS and other spectra.

### BRIEF SUMMARY

A structure of the present invention, which can be used to support a sample for examination by SERS and other spectrometric techniques, comprises a support layer, a metal island layer, and an adhesive layer attaching the metal island layer to the support layer, the adhesive layer allowing movement, and in some instances chemical restructuring, of the metal islands toward increased proximity upon application and drying of an analyte containing solution. This structure is herein referred to as an adaptive metal film to distinguish it from the static metal films of the prior art. The term metal island is being adopted to indicated the discontinuous character of the adaptive metal film. The adhesive layer can be integral with the support layer. The adhesive layer can be a biocompatible material that will permit movement of the metal islands into closer proximity upon exposure to an analyte containing solution, but will securely fix the metal islands stabilized by the analyte with respect to the support layer upon drying of the solution. The adhesive layer is desirably a material that, in areas not contacted by an analyte containing solution, will allow the removal of the metal islands by washing with a suitable buffer solution. A suitable material for the adhesive layer is vacuum deposited silica. Other suitable materials include alumina, titanium oxides, chromium oxides, zinc oxide, and mixtures of one of those with titanium or chromium, e.g., an SiO₂—Ti mixture.

The support layer can be any useful material including a dielectric material, such as glass. The support layer can comprise a bulk metal layer. The bulk metal layer can be located between a dielectric support layer and the adhesive layer. The bulk metal layer is desirably fabricated from highly conductive material typically used for optical mirrors and desirably has a mirror like surface. Suitable materials for the bulk metal layer are silver, gold, aluminum, copper coated with sub-layers of titanium or chromium.

The adaptive metal films can be formed on a dielectric substrate under vacuum evaporation with an electron beam or other vacuum evaporator at an at least moderately hard vacuum. The metals that can be used in the adaptive metal films of the present invention include copper, gold, silver, platinum and palladium, but silver is the preferred metal. The adaptive character of the metal films of the present invention is believed to involve a competition of two processes. A solution of a biomolecule, such as a protein, in a buffered saline may etch metal particle surfaces of the adaptive metal films sufficiently to allow movement of the metal particles relative to the underlying adhesive layer, which are subsequently stabilized interaction with an analyte such as a biomolecule. Since the metal island surface tends to be oxidized during shelf time, a metal surface de-oxidation is also involved in the process of the metal island restructuring. The buffered saline is believed to enhance the stabilization of the metal particles on surfaces of the analyte that may be most suitable for interaction by charge-transfer complexing or other mechanisms. This analyte directed movement of the metal islands leads to significantly enhanced spectral signals particularly with SERS. The optimal proximity is achieved if about a monolayer of an analyte is situated between the stabilized metal particles.

Thus, one aspect of the present invention is a method for preparing an analyte biomolecular sample for collection of spectra data including the steps of depositing the sample in a suitable solution on an adaptive metal film, and allowing the sample to move the metal islands of the film into spectral enhancing proximity during drying of the sample. The metal islands of the adaptive metal film can be considered as initially only modestly bonded to the adhesive layer. Subsequent to exposure to a suitably buffered solution of a analyte of interest, the metal islands that couple to the analyte become more tightly bonded to the adhesive layer so that after drying, any subsequent rinsing by a buffer capable of releasing the nonreacted metal islands for the adhesive layer is unable to release the analyte coupled islands from the adhesive layer. A mixture of an analyte with another molecule can be used to stabilize the metal particle surface. Suitable molecules are polymers like polyvinylpyrrolidone (PVP) that act as stabilizations agents along with the analyte and provide a relatively weak SERS signal. In such a mixture, the concentration of the analyte can be much lower than needed to perform surface restructuring and stabilization.

The adaptive metal films of the present invention can be fabricated to have a range of evaporation parameters that allow for fine rearrangement of their local structure when exposed to various biomolecular solutions. By selecting a suitable adaptive metal film, the adsorbed biomolecules may experience little if any significant changes in conformation during deposition and drying, thus enabling the study of such biomolecules in their natural state. Further the metal-analyte combinations that result from the use of the adaptive metal films of the present invention resist washing and provide enhanced spectral response. Thus, through the use of the adaptive metal films of the present invention one is able to study biomolecules in close association with metal particles naturally located in structures that improve SERS, resist washing, and preserve biomolecule conformation. This is particularly useful when studying large proteins and protein microarrays.

While the following discussion will present a number of examples of the use of the adaptive metal films of the present invention, the examples are not intended to be limiting in any way, but merely exemplary of the range of utility of the present invention.

### BRIEF DESCRIPTION OF THE DRAWINGS

FIG. 1a is a schematic side elevation of an adaptive metal film of the present invention.

FIG. 1b is a schematic side elevation of another adaptive metal film of the present invention.

FIG. 2a is a schematic side elevation of an adaptive metal film of the present invention, which could be an adaptive metal film of either FIG. 1a or FIG. 1b.

FIG. 2b is a schematic view similar to FIG. 2a of an analyte solution droplet on the adaptive metal film.

FIG. 2c is a schematic view similar to FIG. 2a of the analyte and silver islands subsequent to the drying of the solution.

FIG. 3a is a photomicrograph of an adaptive metal film of the present invention.

FIG. 3b is a photomicrograph similar to FIG. 3a subsequent to deposition of the analyte and after rinsing with a suitable buffer.

FIG. 4a is a photograph of an adaptive metal film of the presentation following deposit and drying of a number of analyte containing droplets.

FIG. 4b is a photograph of the same adaptive metal film subsequent to a post drying rinsing step.

FIG. 5 is a graph comparing the extinction intensity in relation to wavelength of an adaptive metal film taken from regions inside and outside an analyte containing droplet.

FIG. 6 is an atomic force microscope image of a adaptive metal film of the present invention before (left) and after (right) the deposition and drying of an analyte containing solution.

FIG. 7 is a high resolution spectrum of the Ag 3 region derived from X-ray photoelectron spectroscopy of an adaptive metal film of the present invention.

FIG. 8a is a superposition of thirty SERS spectra of the fAb monoclonal antibody on an adaptive metal film of the present invention, fifteen before and fifteen after being incubated with fBAP.

FIG. 8b is a superposition of thirty SERS spectra of the fAb monoclonal antibody on an adaptive metal film of the present invention, fifteen before and fifteen after being incubated with BAP.

FIG. 9a is a superposition of eighteen SERS spectra of fBAP on an adaptive metal film of the present invention, nine before and nine after being incubated with fAb.

FIG. 9b is a plot of the two averages of each of the nine spectra shown in FIG. 8a.

FIG. 10 shows detection by chemiluminescence (left) and fluorescence (right) on an adaptive metal film of the present invention

FIG. 11a is a SERS spectra collected at 568 nm incident laser wavelength for human insulin and insulin lispro on an adaptive metal film of the present invention.

FIG. 11b is a plot of the difference between the SERS spectra shown in FIG. 11a highlighting the differences between the two isomers.

FIG. 12 is a graph of the relative intensities of a SERS signal from a common sample placed on adaptive metal films of FIGS. 1a and 1b in relation to plain glass.

### DESCRIPTION OF THE PREFERRED EMBODIMENTS

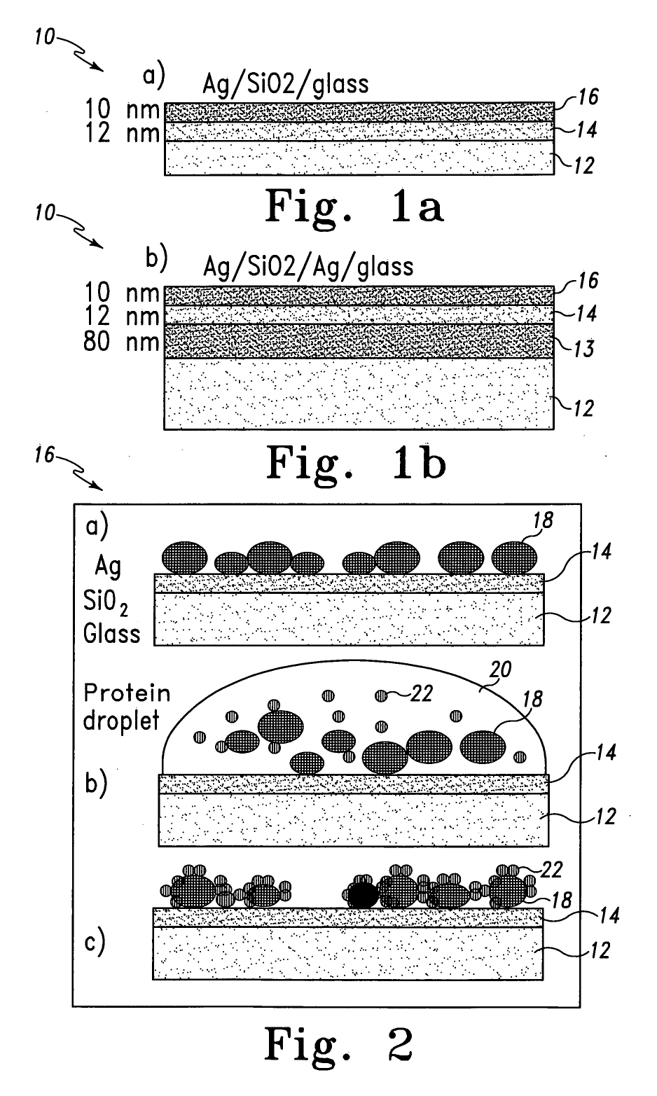

The layered structures of the present invention can be formed on a dielectric substrate such as glass. One structure of an adaptive metal film 10 of the present invention is shown in FIG. 1a to comprise a dielectric substrate 12 of glass covered entirely by an adhesive layer 14 of SiO₂ on top of which is a sparse metal island layer 16, which can be thought of as a nanostructured semicontinuous metal film. The dielectric substrate 12 can be of any thickness required to support the adaptive metal film structure as a whole. The adhesive layer 14 is more than 5 nm and typically about 8-12 nm thick. The adhesive layer 14 can be formed of a material that under certain conditions will release the sparse metal layer 16. Depending on the material selected for the adhesive layer 14, the sparse metal layer 16 can be between about 3 and 25 nm thick. The sparse metal layer 16 can be about 8-13 nm thick in the case of silica. The SERS signals for materials deposited on the adaptive metal films of the present invention can be materially increased by including a bulk metal layer 13 that is situated between the dielectric substrate 12 and the adhesive layer 14 as shown in FIG. 1b. The bulk metal layer 13 is preferably mirror-like in surface character and can be thicker than the combined adhesive layer 14 and sparse metal layer 16. The bulk metal layer 13 can be about 40-300 nm thick, and is preferably about 80 nm in thickness. The bulk metal layer 13 provides an additional enhancement of the local fields caused by interaction between the metal particles of the sparse metal layer 16 and their images in the bulk layer 13 so that SERS signal intensity may increase by as much as 4 or 5 times relative to the SERS signal derived from an adaptive metal film of FIG. 1a.

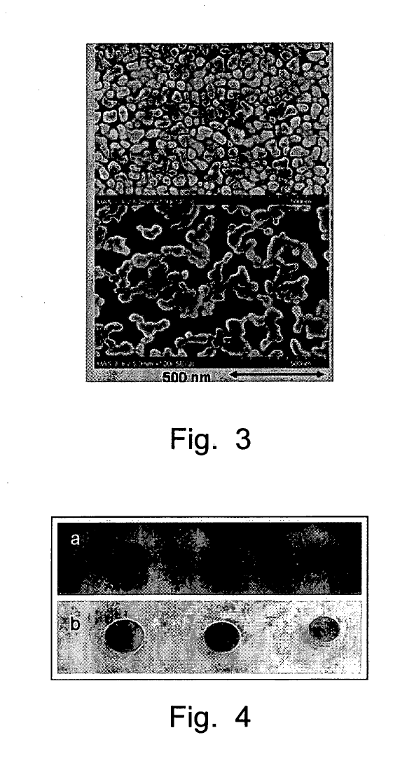

The substrate 12 can be a clean glass slide of the type typically used for light microscopy. The glass slide can be cleaned using a number of steps. For example, a glass slide can be washed multiple solvent rinses and then soaked in a piranha (H₂O₂:3H₂SO₄) acid bath, rinsed in 18 MΩ deionized water, and dried with pressurized gaseous nitrogen. The cleaned glass slide can then placed in an electron beam evaporator with an initial pressure inside the system of about 10⁻⁷ Torr. The glass slide can be covered with a base metal layer as shown in FIG. 1b. The substrate is then covered by about a 10 nm layer of SiO₂. Next, an 8-13 nm Ag layer is deposited at a rate of about 0.05 nm/sec. During the silver deposition process, small isolated metal granules form first on the silica surface. As the silver coverage increases, the granules coalesce, resulting in various sizes of silver particles and their aggregates. The resulting adaptive metal film 10 is schematically shown in FIG. 2a, but the adaptive metal film 10 of FIG. 1b could be substituted. A plan view of the adaptive metal film 10 is shown in FIG. 3a, which is a photomicrograph showing metal islands of about 50 nm average diameter and about 10 nm average thickness.

The addition of a biomolecule of interest to an adaptive metal film 10 of the present invention can be accomplished by initially forming a suitable solution of the biomolecule. For example, a 0.5 μM solution of an analyte, such as bacterial alkaline phosphatase (BAP), can be prepared. Aliquots of about 2 μL of the analyte solution can be deposited, either manually or by a suitable dispensing head, on the adaptive metal film 10 in separate droplets 20. A solution droplet 20 is schematically shown in FIG. 2b containing the analyte 22 and illustrating the at least partial solution of some of the silver islands 18 within the droplet 20 so that the islands 18 are able to move relative to the substrate 12 during the drying of the solution. The droplets 20 can be dried at ambient room temperature. The adaptive metal films 10 can be subjected to moderate vibration or shaking during the drying process to encourage movement of the silver islands 18. The drying can take two hours or more when dried in this manner. The resulting dried area of the adaptive metal film 10 is schematically illustrated in FIG. 2c with particles of the analyte 22 in direct contact with the silver islands 18. Following the drying step, the adaptive metal film 10 can be rinsed with a suitable buffered solution. The rinsing step can remove silver islands 18 that were not contacted by or connected to the analyte.

A plan view of an adaptive metal film 10 subsequent to the rinsing step is shown in FIG. 3b, while FIG. 3a shows the same adaptive metal film prior to deposition of the analyte solution. The images shown in FIGS. 3 were obtained using a field emission scanning electron microscope (FE SEM). The images show metal nanoparticles and their aggregate islands in white, while the dielectric material is dark gray or black. A comparison of FIG. 3b with FIG. 3a shows some aggregation of metal islands has occurred during the drying step. FIG. 3b shows the nanoscale restructuring inside the biomolecule solution spot where groups of closely spaced metal nanoparticles coalesce. This contrasts with areas outside the biomolecule solution spot where rather disintegrated particles are typical. Decreases in the integrated extinction and in the metal filling factor both suggest a decrease of the silver mass thickness caused by biomolecule deposition, indicating that some of the metal nanoparticles have been dissolved in the solution. It will also be seen that the biomolecules themselves stabilize the metal film sufficiently to permit washing of the adaptive metal film with a buffered saline solution. The biomolecules themselves play a key role in forming stable complexes with the metal particles.

FIG. 4a is a plan view on a much larger scale than FIG. 3 showing a number of distinctly separated analyte solution droplet areas on a single adaptive metal film 10 after drying but prior to the rinsing step. FIG. 4b shows the same adaptive metal film subsequent to the rinsing step. The images shown in FIG. 4 were obtained using a common digital optical camera. By varying the analyte and buffer concentrations, one can determine that both concentration factors play a role in the formation of uniform, stabilized analyte spots. By lowering the biomolecule concentration by roughly a factor of 10-20, the spots become almost transparent indicating a lack of interaction between any biomolecule and the sparse metal film layer 16. Any attempt to deposit biomolecules in the absence of a buffer also leads to no visible change to the adaptive metal film 10 indicating that the buffer is apparently necessary to trigger the interaction between the adaptive metal film and the biomolecules. A comparison of metal substrates with and without the adhesive layer shows that adhesion of silver particles directly on glass is poor. The substitution of a very high adhesion layer, such as a titanium layer, so stabilizes the metal islands of the top layer that the desired structural adaptation of the metal islands does not occur and typically little or no SERS spectra is observed. While biomolecules are of greatest interest, the same adaptive metal film restructuring and stabilization can occur with synthetic polymers such as PVP.

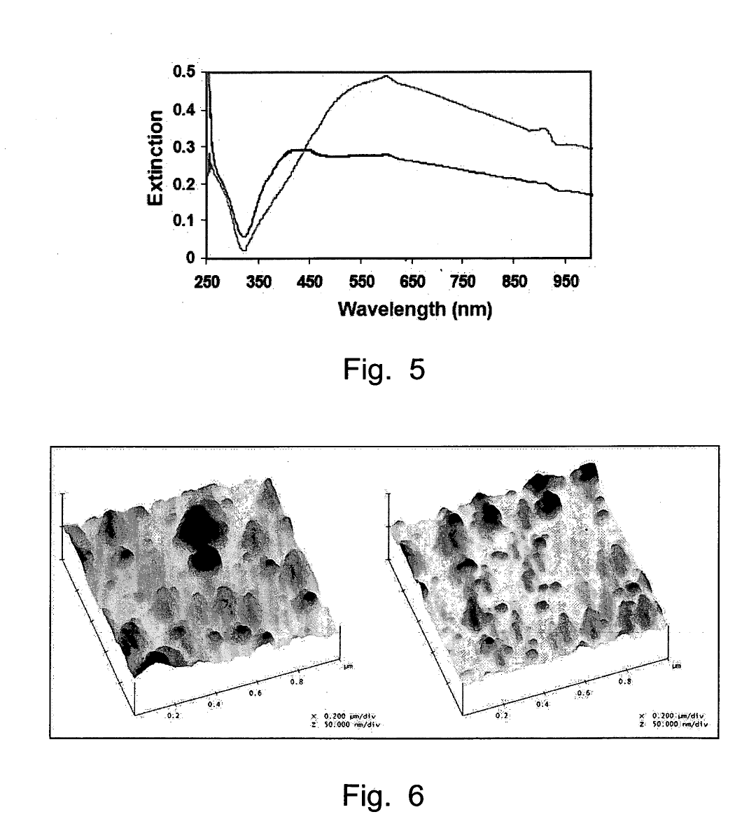

Light impinging on a semicontinuous metal film will undergo transmission, reflection, or absorption, with a small percentage, typically less than 5%, experiencing scattering. By measuring the intensity of light in each of these processes at different frequencies, one can judge whether the film structure supports resonant plasmon modes. At the frequencies where resonances occur, absorption increases. FIG. 5 compares the intensity of the absorption (extinction) spectra derived from insulin deposited on an adaptive metal film of the present invention in relation to spectra derived from the same adaptive metal film, but outside the insulin deposition spots. The absorbance of an adaptive metal film of the present invention can be seen to increase upon deposition of a suitably buffered analyte solution with a maximum extinction occurring around 500 nm with a broad wing into the longer wavelengths.

While the FE SEM images of FIG. 3 provide a picture of the lateral structure of an adaptive metal film of the present invention, atomic force microscopy (AFM) can provide additional information about the vertical structure of the adaptive metal film. FIG. 6 shows a typical AFM height profile for an adaptive metal film of the present invention before (left) and after (right) deposition of a suitably buffered analyte containing droplet. The AFM analysis indicates that the particle height is typically less than their lateral plane size. The example illustrated in FIG. 6 shows a maximum height of about 30 nm with a RMS deviation from the mean in the range of about 5-7 nm. At the same time the adaptive metal film is uniform on the scale of micrometers and higher so that the film is very homogeneous within the typical diameter of a SERS excitation laser spot of about 80 to 100 μm.

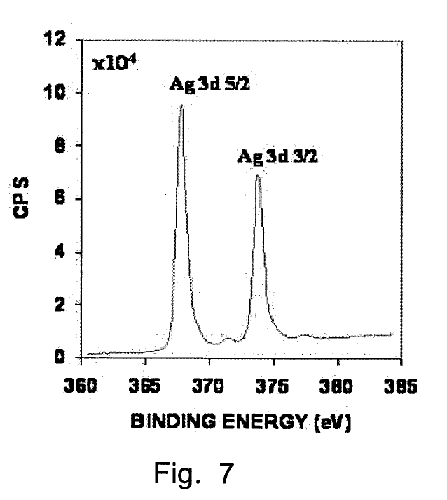

The elemental species on a sample surface can be analyzed using X-ray photoelectron spectroscopy (XPS). This technique is capable of probing roughly 10 nm into a sample surface, which is the approximate thickness of the metal island layer of an adaptive metal film of the present invention (and any biomolecules deposited onto the film surface). By measuring the kinetic energy of the photoelectrons at a given photon energy (typically 1486.6 eV), one can detect the binding energy spectrum. The binding energy peaks are characteristic for each element, while the peak areas can be used to determine the relative composition of the sample surface. The shape of each peak and the binding energy can be slightly altered by the chemical state of the emitting atom. Hence, XPS can provide chemical bonding information as well. FIG. 7 is a representative XPS high energy resolution spectrum of the Ag 3d region of an adaptive metal film of the present invention where the metal island layer is formed of silver. An important result of this study is that the silver on the substrate is still in a metal state without oxidation even 2 to 3 weeks after fabrication. However, by about eight weeks after fabrication the metal is predominately in an oxidized state. The presence of metal-state silver indicates that the adaptive metal films of the present invention are reasonably stable over time, which is helpful for production and storage of the structures. Deposition of a buffered protein solution typically causes the silver to at least partially deoxidize allowing use of long stored adaptive metal films of the present invention.

The adaptive metal films 10 of the present invention are suitable for use in a number of testing situations. For example, the adaptive metal films of the present invention can be used to probe antigen-antibody binding. This can be accomplished by depositing and immobilizing a monoclonal antibody or a corresponding antigen on an adaptive metal film. Typically, 2 μL of 0.5 μM antibody solution form a spot of about 2 mm diameter after drying overnight. The non-adherent metal particles of the adaptive metal film are then removed by washing with a buffered solution and deionized water to reveal immobilized protein-adapted aggregates representing antibody (or antigen) in a small array. The specific proteins used in the development of the process included the anti-FLAG M2 monoclonal antibody (fAb) and the bacterial alkaline phosphatase/C-terminal FLAG-peptide fusion (fBAP). Proteins for control experiments included the bacterial alkaline phosphatase (BAP) without the FLAG peptide, which was generated by enterokinase cleavage. Subsequent incubation of the protein-adapted aggregates With antigen (or antibody) was conducted. The nonspecifically-bound material was removed by washing with a standard buffer solution for Western blotting (TBS/Tween-20) followed by rinsing five times with deionized water. SERS spectra of the immobilized fAb (or fBAP) were compared before and after reaction with the cognate antigen (or antibody) partner.

The SERS spectra can be collected with a variety of known instruments. The spectra recorded herein were collected with a four-wavelength Raman system that included an Ar/Kr ion laser (from Melles Griot), a laser band-path holographic filter (to reject plasma lines) and two Super-Notch Plus filters (from Kaiser Optical Systems) to reject Rayleigh scattering, focusing and collection lenses, an Acton Research 300i monochromator with a grating of 1200 grooves/mm, and a nitrogen-cooled CCD (1340×400 pixels from Roper Scientific). SERS spectra were typically collected using a laser beam excitation wavelength of 568.2 nm with normal incidence and 45° scattering. An objective lens (f/1.6) provided a collection area of about 180 μm². Collected light was delivered to the spectrometer via a fiber bundle. The spectral resolution was about 3 cm⁻¹.

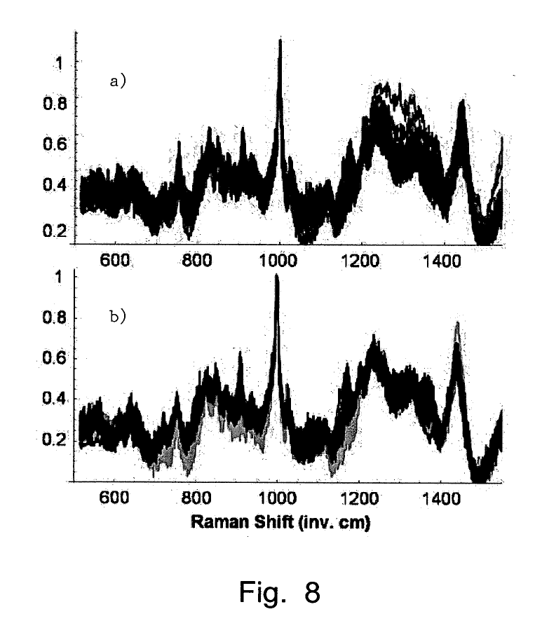

The immobilized fAb/metal clusters that result from deposition and drying of the fAb on an adaptive metal film yielded reproducible SERS results, and representative spectra are shown in FIGS. 8a and 8b by the dark spectral core. Upon incubation of the immobilized fAb with fBAP (0.75 nM) the spectral changes shown in grey in FIG. 8a were observed with the most dominant features appearing in the 1200-1400 cm⁻¹ region of the spectrum. Incubation of the immobilized fAb with BAP (0.67 nM) resulted in almost no spectral changes as shown by the modest grey features in FIG. 8b. Thus, a specific antibody-antigen binding event can be detected using the adaptive metal films of the present invention. The spectral variations are attributable to a spatial variation in the distribution of antigen binding. Repeated formation of specific antibody-antigen complexes on a number of adaptive metal films result in the same characteristic spectral change in the 1200-1400 cm⁻¹ region.

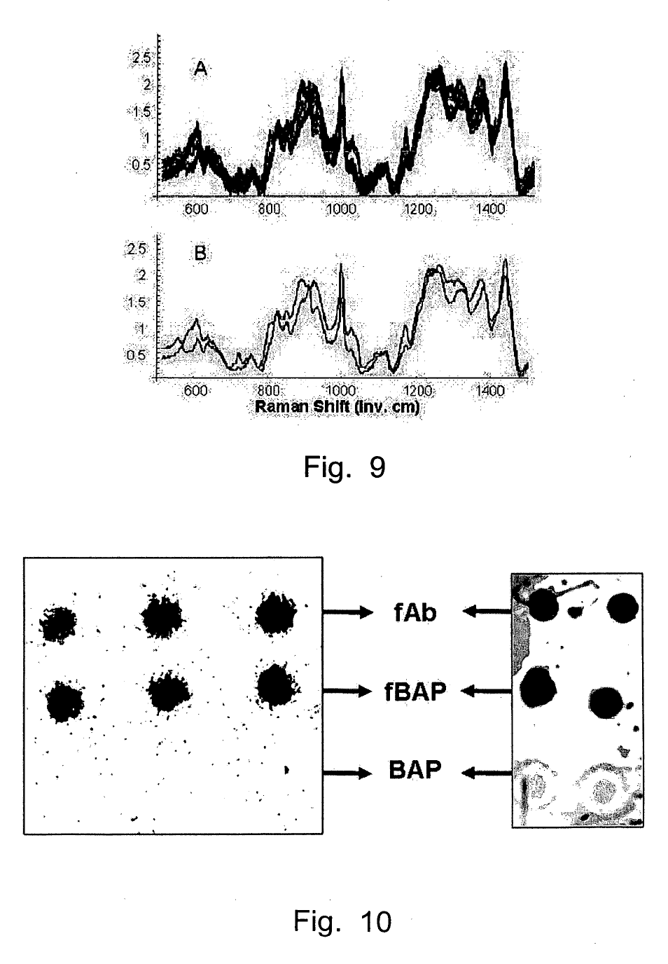

Further validation of the detectability of such interactions is observed by first depositing and drying a fBAP solution (0.5 μM in TBS buffer) on an adaptive metal film 10 of the present invention. The washed and rinsed fBAP surface was then incubated with fAb at a concentration of about 4 nM. The results of the SERS measurements before and after the incubation are shown in FIGS. 9a and 9b. The changes that are most evident are the group of peaks centered at around 900 cm⁻¹, where a triple peak appears instead of the double peak present before incubation with the antibody. The intensity of the peak at about 1000 cm⁻¹ is significantly decreased, while the intensity of the peak at about 1280 cm⁻¹ is increased. For both of these experiments, the signal from the first layer of protein dominates the SERS spectra observed from the immune complex. Therefore the spectra in FIGS. 8 and 9 are significantly different, with the spectra of FIG. 8 being primarily the SERS spectrum of fAb, while the FIG. 9 spectra primarily represents the fBAP spectrum. In both cases, binding with proteins of the second layer results in detectable and reproducible changes in the SERS spectrum of the first layer. The fact that the first layer dominates the observed SERS spectra indicates the important role of the surface enhancement achieved with the initial reaction with the adaptive metal film of the present invention.

The nanostructured adaptive metal films of the present invention can also be used with other detection methods such as chemiluminescence and fluorescence to study the same deposit. For example, alternative detection methods can be used to validate the integrity of the fAb-fBAP binding events on the adaptive metal film, and to assess the utility of the protein-adapted clusters for applications in protein binding assays. For detection by chemiluminescence, fAb, fBAP and BAP were each deposited on an adaptive metal substrate at equal concentrations and sequentially reacted with fAb and HRP-conjugated anti-mouse IgG secondary antibody. The specificity of the reactions shown in FIG. 10a indicates that fAb and the secondary antibody are functional on these adapted surfaces. Similar experiments were performed for fluorescence detection except that the fAb, fBAP and BAP were arrayed and probed with Cy3-conjugated fAb. The specific interaction of the Cy3-conjugated fAb is observed with the fBAP. These results are consistent with those observed using the secondary antibody/chemiluminescence detection strategy. In both cases, the results indicate that the functional properties of immobilized proteins are largely preserved on the adaptive metal films of the present invention.

A study of insulin and insulin analogs demonstrates the discrimination capacity the adaptive metal substrates of the present invention. It is well known that insulin is composed of two peptide chains referred to as the A and B chains. The two chains are linked together by two disulfide bonds, and an additional disulfide is formed within the A chain. The A chain consists of 21 amino acids, while the B chain consists of 30 amino acids. Insulin monomer is the active form of the hormone. Insulin exists as a monomer in solution at neutral pH and at physiological concentrations (about 1 ng/mL). Hydrogen bonding between C-termini of the B chains in solution results in a tendency to form dimers of human insulin molecules. Anti-parallel-pleated-sheet interactions are involved in the formation of insulin dimers. Additionally, in the presence of zinc ions, insulin dimers associate into hexamers. These interactions have very important clinical effects because monomers and dimers readily diffuse into blood, whereas hexamers diffuse very poorly. As a result, absorption of insulin preparations containing a high proportion of hexamers is strongly delayed.

This has stimulated development of a number of recombinant insulin analogs. The first of these molecules, which is called insulin lispro, is engineered so that lysine and proline residues on the C-terminal end of the B chain are interchanged in their positions. Thus lispro is Lys(B28) Pro(B29) human insulin analog, having the identical chemical composition and molecular weight with normal human insulin. It is very hard or impossible to distinguish the two insulins with convention protein analysis techniques, such as mass spectroscopy and chromatographic separation. The lispro modification minimizes the tendency to form dimmers and hexamers but does not alter receptor binding. As a result, insulin lispro is a rapidly acting, parenteral blood glucose-lowering agent.

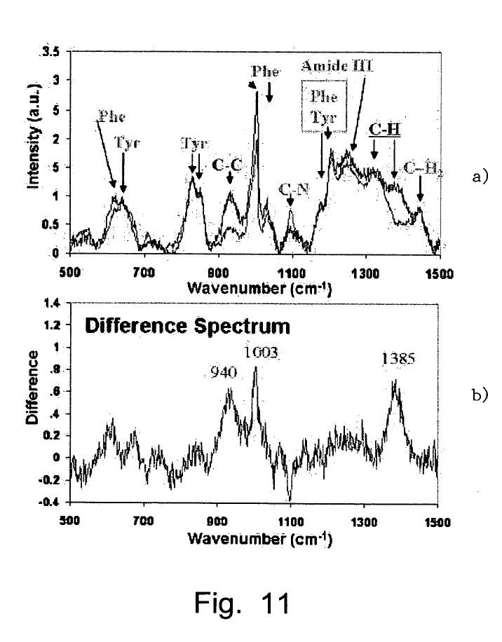

The comparative study of human insulin and lispro was conducted on adaptive metal films of the present invention previously described in connection with FIG. 7. A 2-4 μL droplet of 1 μM concentration insulin or lispro solution was deposited on an adaptive metal film and then allowed to dry resulting in a surface density of about 80±20 fmol/mm². A representative SERS spectrum was collected from the central part of each droplet deposit. Both SERS spectra are shown in FIG. 11a, while FIG. 11b shows the difference between the two spectra shown in FIG. 11a. The human insulin spectrum has more intense peaks at 94, 1003, 1385, 1605 cm⁻¹, and in the 1650-1660 cm⁻¹ band. The difference curve is more pronounced at low excitation laser intensity and power (1 mW), but becomes less pronounced under exposure to higher power. FIG. 11b graphically demonstrates that SERS spectra collected from adaptive metal films of the present invention can clearly distinguish between native human insulin and lispro. The observed difference is highly reproducible and was obtained for different droplet deposits and different adaptive metal films.

The observed difference can be attributed to conformational differences in the two biomolecules. The solutions employed in the development of the spectra shown in FIG. 11 contained Zn and Cl⁻¹. The presence of chloride ions results in the T→R transition. Typically the transition is accompanied by the hexamer formation in the presence of zinc. While the exact balance ratio is difficult to determine, the SERS difference spectra indicate that a preferred conformation state for human insulin is the R-state, while for insulin lispro the preferred conformation is the T and/or R^{f} state. This conclusion is in agreement with X-ray crystallographic studies that indicate insulin lispro crystallizes as a T₃R^{f}₃ hexamer. The presence of the zinc and chloride ions appears to stabilize the R₆ state of human insulin hexamers resulting in the observed spectral differences. Further the presence of the small amount of zinc ions does not appear to affect the mobility of the metal islands in the adaptive metal film during the solution deposition and drying. It can also be seen that the observed SERS difference spectra collected from adaptive metal films can provide important information about conformation states of biomolecules that are hard to distinguish with conventional methods. The adaptive metal films of the present invention exhibit a high SERS sensitivity in detection and characterization of proteins down to the sub-monolayer level.

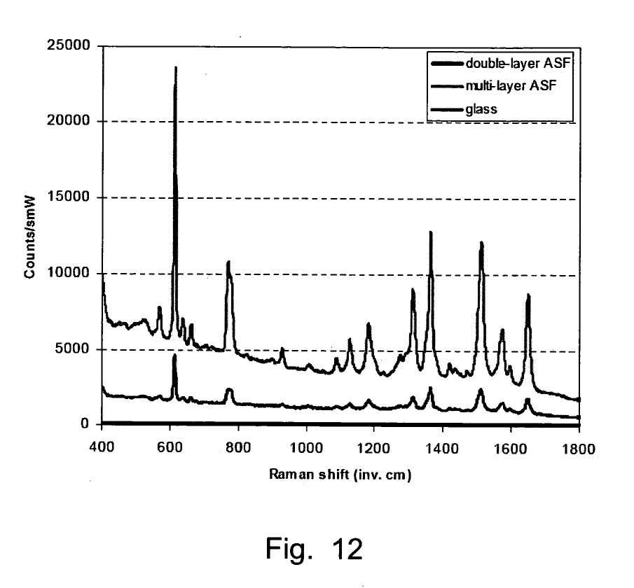

The SERS signals for materials deposited on the adaptive metal films of the present invention can be materially increased by including a bulk metal layer 13 that is situated between the dielectric substrate 12 and the adhesive layer 14 as shown in FIG. 1b. The bulk metal layer 13 provides an additional enhancement of the local fields caused by interaction between the metal particles of the sparse metal layer 16 and their images in the bulk layer 13 so that SERS signal intensity may increase by as much as 4 or 5 times relative to the SERS signal derived from an adaptive metal film of FIG. 1a. The localized plasmon resonance supported by the islands forming the sparse metal layer 16 exhibit a frequency shift when placed in close proximity to a conducting surface presented by the bulk layer 13. FIG. 12 shows the relative increment of the SERS intensity for a sample consisting of an antibody (anti-human interleukin 10) incubated with R6G on an adaptive metal film of FIG. 1a and FIG. 1b in comparison to plain glass. While the performance difference is readily apparent, a comparison of the specific intensities (in counts per second per mW) of a characteristic peak provides some quantitative measure of the differences as shown in Table 1.

TABLE 1
SERS Signal Comparison for Two Adaptive Metal Films

| | FIG. 1a film | | FIG. 1b film | |
|---|---|---|---|---|
| Insulin | Max 7 | c/smW | 28-38 | c/smW |
| Antibody | 20-25 | c/smW | 80-100 | c/smW |
| Antibody with R6G | 3500 | c/smW | 16000 | c/smW |

The nanostructured adaptive metal films of the present invention exhibit clear advantages over static structure SERS substrates. The adaptive feature of the films of the present invention appears to produce cavity sites created by two or more metal particles or islands, the cavity sites being filled with, and at least to some extent defined by, the analyte of interest. The adaptive metal films of the present invention experience fine restructuring under analyte solution deposition such that the conformation and functionality of biomolecular analytes are largely preserved.

The foregoing detailed description should be regarded as illustrative rather than limiting, and the following claims, including all equivalents, are intended to define the spirit and scope of this invention.

1. An adaptive surface for supporting an analyte to be examined, the surface comprising:
a support layer, a metal island layer, and an adhesive layer by which the metal island layer is attached to the support layer, the adhesive layer and metal island layer being interactive with an analyte containing solution to permit movement of at least some of the islands on the adhesive layer into increasingly close proximity during drying of the analyte solution.

2. The adaptive surface of claim 1 wherein the support layer consists essentially of a dielectric material.

3. The adaptive surface of claim 1 wherein the support layer comprises a bulk metal layer fixed on top of a dielectric layer.

4. The adaptive surface of claim 2 or 3 wherein the dielectric layer comprises a glass.

5. The adaptive surface of claim 3 wherein the bulk metal layer consists essentially of a highly conductive metal with a mirror like surface.

6. The adaptive surface of claim 5 wherein the metal in the bulk metal layer is selected from the group consisting of silver, gold, aluminum, copper, optionally including sub-layers of titanium or chromium.

7. The adaptive surface of claim 3, 5 or 6 wherein the bulk metal layer has a thickness of between about 40 to 300 nm

8. The adaptive surface of claim 7 wherein the bulk metal layer has a thickness of between about 80 nm.

9. The adaptive surface of claim 1 or 2 wherein the metal in the metal island layer is selected from the group consisting of silver, copper, platinum, palladium, and gold.

10. The adaptive surface of claim 1 or 3 wherein the adhesive layer consists essentially of a material selected from the group of silica, alumina, titanium oxides, chromium oxides, zinc oxide, and mixtures of one of the preceding with titanium or chromium, the selected material being vacuum evaporated on the support layer.

11. The adaptive surface of claim 1 or 3 wherein the adhesive layer has a thickness of between about 5 to 500 nm.

12. The adaptive surface of claim 11 wherein the adhesive layer has a thickness of between about 8 to 12 nm.

13. The adaptive surface of claim 9 wherein the metal island layer comprises silver islands vacuum evaporated on the adhesive layer.

14. The adaptive surface of claim 1 or 3 wherein the metal island layer has a thickness of between about 3 to 25 nm.

15. The adaptive surface of claim 14 wherein the metal island layer has a thickness of between about 8 to 13 nm.

16. A method of collecting spectral data from an analyte comprising the steps of:
a) providing an adaptive surface comprising a metal island layer adhered to a support layer by an adhesive layer,
b) depositing a solution of the analyte on the adaptive surface, any
c) allowing the analyte solution to interact with the adaptive surface so that the metal islands move into increasing proximity, and
d) drying the sample solution to stabilize the metal islands in contact with the analyte, thereby providing enhanced spectral response of the analyte.

17. The method of claim 16 further comprising the step of washing the adaptive surface with a solution to remove metal everywhere except the metal islands in contact with the analyte.

18. The method of claim 16 further comprising the step of washing the adaptive surface with a solution to remove any surface chemistry reaction products except the metal islands in contact with the analyte.

19. The method of claim 16 or 17 or 18 further comprising the step of recording a SERS spectrum for the sample on the adaptive surface layer.

20. The method of claim 19 further comprising the steps of applying a second analyte solution to the adaptive surface, incubating the analytes, and recording a second SERS spectrum for the combined analytes.

21. The method of claim 20 further comprising the step of constructing a difference spectrum by subtracting one of the SERS spectra from the other.

22. The method of claim 21 further comprising the step of comparing points on the difference spectrum to known Raman spectra.
